(19) United States
(12) Patent Application Publication
(10) Pub. No.: US 2006/0000033 A1
(43) Pub. Date: Jan. 5, 2006

(54) Dyeing composition comprising at least one elastomeric film-forming polymer and at least one oxidation dye precursor

(76) Inventors: Isabelle Rollat-Corvol, Paris (FR); Jonathan Gawtrey, Boulogne (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/171,224

(22) Filed: Jul. 1, 2005

Related U.S. Application Data
(60) Provisional application No. 60/616,225, filed on Oct. 7, 2004.

(30) Foreign Application Priority Data
Jul. 1, 2004 (FR) 04 51389

(51) Int. Cl. A61K 8/00

(52) U.S. Cl. 8/405

(57) ABSTRACT

The present disclosure relates to a dyeing composition comprising, in a medium suitable for dyeing, at least one dye precursor and at least one elastomeric film-forming polymer wherein the film obtained by drying this polymer, at ambient temperature and at a relative humidity of 55%±5%, has properties comprising:
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%. This composition makes it possible, for example, to obtain strong colorations that withstand outside agents.

This application claims benefit of U.S. Provisional Application No. 60/616,225, filed Oct. 7, 2004, the contents of which are incorporated herein by reference. This application also claims benefit of priority under 35 U.S.C. § 119 to French Patent Application No. 04 51389, filed Jul. 1, 2004, the contents of which are also incorporated by reference.

The present disclosure relates to a dyeing composition comprising, in a medium suitable for dyeing keratin materials, at least one oxidation dye precursor and at least one film-forming polymer with specific elastomeric characteristics. The present disclosure further relates to a method for dyeing keratin materials, such as keratin fibers, using this composition.

For a long time, many individuals have been seeking to modify the color of their skin, eyelashes or hair, for example, to mask their white hair. To do this, several technologies have been developed.

It is known practice to dye human keratin fibers, such as the hair, with dye compositions containing oxidation dye precursors such as oxidation bases or couplers. Oxidation bases are colorless or weakly colored compounds which, when combined with oxidizing products, give rise, by a process of oxidative condensation, to colored compounds. These dyes are insoluble and are trapped inside the hair fiber.

It is also known that it is possible to vary the shades obtained with these oxidation bases by combining them with color modifiers or couplers. The variety of molecules involved in terms of oxidation bases and couplers makes it possible to obtain a rich palette of colors.

The colorations obtained show good fastness with respect to shampoos. However, the oxidation reaction is carried out by oxidizing products such as aqueous hydrogen peroxide in a basic medium. These oxidizing agents attack the keratin of the hair, the cosmetic and mechanical properties of which can be highly degraded in the case of repeated dyeing, which can result in difficulty in styling or shaping.

It is already known practice to introduce, into some oxidation dye compositions, polymers conventionally used for formulating hair products for styling. It is known practice, for example, to add anionic, amphoteric or non-ionic film-forming polymers that result in the formation of films having a more or less hard and brittle nature.

There is a need, in the dyeing field, for a composition that makes it possible to obtain at least one of strong colorations, in varied shades, that have good fastness over time and withstand outside agents such as light, shampoo or sweat, while at the same time preserving the quality of the keratin material, for example, its ease of styling, such as satisfactory untangling, softness, and a pleasant, non-tacky appearance, and ease of use. There is also a need for dyeing compositions capable of forming on these keratin materials a flexible film that follows the movements of the skin or the head of hair, without any effect of tautness or heaviness, or any rigid sensation.

Thus, disclosed herein is a dyeing composition comprising, in a medium suitable for dyeing, at least one dye precursor and at least one elastomeric film-forming polymer wherein the film obtained by drying this polymer, at ambient temperature and at a relative humidity of 55%±5%, has properties comprising at least one of:
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%.

This composition makes it possible in some embodiments, for example, to obtain strong colorations that withstand outside agents, while at the same time preserving the integrity of keratin materials and good styling, by the formation of a flexible, non-brittle film on human keratin materials, that follows their movements.

As used herein, the term “at least one” elastomeric film-forming polymer means one or more (2, 3 or more) elastomeric film-forming polymers.

The term “dye precursors” means any non-colored molecule that, under the action of an oxidizing agent, provides a colored species which colors keratin materials.

Further disclosed herein is a method for dyeing keratin materials, such as keratin fibers, using the composition disclosed herein.

Even further disclosed herein is the use of this composition for dyeing keratin materials, for example, keratin fibers, such as the hair.

As used herein, the term “film obtained by drying at ambient temperature (22° C.±2° C.) and at a relative humidity of 55%±5%” means the film obtained, under these conditions, from a mixture comprising 6% of active material (A.M.) of the at least one elastomeric film-forming polymer in a mixture of 30% by weight of ethanol and 70% by weight of water, relative to the total alcohol+water weight, the amount of mixture being adjusted so as to obtain, in a Teflon matrix, a film 500 μm±50 μm thick. The drying is continued until the weight of the film no longer changes, which lasts approximately 12 days. The film-forming polymers that are soluble or partially soluble in ethanol are tested in ethanol alone. The other polymers are tested in water alone, in soluble or dispersed form. The relative humidity can readily be determined by one of ordinary skill in the art using the known techniques.

As used herein, the elongation at break and the recovery rate are evaluated by the tests described below.

To carry out the tensile tests, the film is cut into rectangular test pieces 80 mm long and 15 mm wide.

The tests are carried out on a device sold under the name Lloyd or sold under the name Zwick, under the same temperature and humidity conditions as for the drying, i.e., a temperature of 22° C.±2° C. and a relative humidity of 55%±5%.

The test pieces are drawn at a speed of 20 mm/min and the distance between the jaws is 50±1 mm.

To determine the instantaneous recovery (Rᵢ), the following procedure is carried out:
the test piece is drawn by 150% (εₘₐₓ), i.e., 1.5 times its initial length (I₀),
the stress is released by applying a return speed equal to the tensile speed, i.e., 20 mm/min, and the percentage elongation of the test piece is measured, after return to a zero constraint (εᵢ).

The % instantaneous recovery (Rᵢ) is given by the formula below:
Rᵢ=((εₘₐₓ−εᵢ)/εₘₐₓ)×100

To determine the recovery at 300 seconds, the test piece, having been subjected to the above operations, is maintained at zero stress for a further 300 seconds, and its percentage elongation (ε₃₀₀ₛ) is measured.

The % recovery at 300 seconds (R₃₀₀ₛ) is given by the formula below:
R₃₀₀ₛ=(εₘₐₓ−ε₃₀₀ₛ)/εₘₐₓ)×100

For example, the at least one elastomeric film-forming polymer of the composition disclosed herein, optionally combined with at least one auxiliary film-forming agent, is such that it forms, under the conditions of the tests above, a film having an elongation at break ranging from 800% to 3000%; an instantaneous recovery ranging from 75% to 100%; and a recovery at 300 seconds ranging from 85% to 100%.

In the compositions disclosed herein, the at least one elastomeric film-forming polymer is present in an amount ranging, for example, from 0.05% to 20% by weight, such as from 0.1% to 15% by weight, and further, such as from 0.25% to 10% by weight, relative to the total weight of the composition.

For example, the at least one elastomeric film-forming polymer is chosen from polyurethanes, polyvinyl alcohols, and polymers comprising at least one (meth)acrylic unit. It may be in the form of a homopolymer or a copolymer. In one embodiment, it is in a non-crosslinked form in the composition.

Further for example, the at least one elastomeric film-forming polymer that is useful herein is soluble or hydrodispersible in an aqueous or aqueous-alcoholic medium. In one embodiment, the at least one elastomeric film-forming polymer is soluble at at least 10 g of active material in 90 g of aqueous or aqueous-alcoholic medium (comprising 70% by_ weight of water and 30% by weight of ethanol), at ambient temperature and atmospheric pressure.

In one embodiment, the film formed from the composition disclosed herein has a low water sensitivity, for example, in an atmosphere with a relative humidity ranging from 30% to 80%, i.e., the film keeps its elastomeric properties for several hours. It is flexible and non-brittle and correctly follows the movements of the skin and/or the head of hair. For example, at a relative humidity ranging from 30% to 80%, the elongation at break of the film obtained does not vary by more than 50% (±400%) and/or its instantaneous recovery does not vary by more than 25% (18.75%). In other words, at a relative humidity ranging from 30% to 80%, the elongation at break of the film obtained ranges from 400% to 1200% and/or its instantaneous recovery ranges from 57% to 93%.

The elastomeric film-forming polymers that are useful in the composition disclosed herein are known in the art. They may, for example, be synthesized according to the method described in French patent application FR 2 815 350.

The dye precursors that are useful in the composition disclosed herein are oxidation bases or couplers that are conventionally used in the field of oxidation dyeing.

As the oxidation bases, mention may, for example, be made of para-phenylenediamines, bisphenylalkylenediamines, para-aminophenols, ortho-aminophenols and heterocyclic bases.

Among the para-phenylenediamines, mention may be made, by way of example, of para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-62 -hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene, 3-hydroxy-1-(4′-aminophenyl)pyrrolidine, and acid addition salts thereof.

Among the para-phenylenediamines mentioned above, mention may be made, for example, of para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N, N-bis(β-hydroxyethyl)-para-phenylenediamine, 2-chloro-para-phenylenediamine, 2-β-acetylaminoethyloxy-para-phenylenediamine, and acid addition salts thereof.

Among the bisphenylalkylenediamines, mention may be made, for example, of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)-tetramethylenediamine, N,N′-bis(4-methylaminophenyl)tetramethylenediamine, N,N′-bis(ethyl)-N,N′-bis(4′amino-3′-methylphenyl)ethylenediamine, 1,8-bis(2,5-diaminophenoxy)-3,6-dioxaoctane, and acid addition salts thereof.

Among the para-aminophenols, mention may be made, by way of example, of para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylpheriol, 4-amino-2-aminomethylphenol, 4-amino-2-(β-hydroxyethyl-aminomethyl)phenol, 4-amino-2-fluorophenol, and acid addition salts thereof.

Among the ortho-aminophenols, mention may be made, by way of example, of 2-aminophenol, 2-amino-5-methylphenol, 2-amino-6-methylphenol, 5-acetamido-2-aminophenol, and acid addition salts thereof.

Among the heterocyclic bases, mention may be made, by way of example, of pyridine derivatives, pyrimidine derivatives and pyrazole derivatives such as 4,5-diaminopyrazole derivatives.

Among the pyridine derivatives, mention may be made of the compounds described, for example, in British patents GB 1 026 978 and GB 1 153 196, such as 2,5-diaminopyridine, 2-(4-methoxyphenyl)amino-3-aminopyridine, 2,3-diamino-6-methoxypyridine, 2-(β-methbxyethyl)amino-3-amino-6-methoxypyridine, 3,4-diamino-pyridine, and acid addition salts thereof.

Other pyridine oxidation bases that are useful herein are the 3-amino-pyrazolo[1,5-a]pyridine oxidation bases, or addition salts thereof, described, for example, in French patent application FR 2801308. By way of example, mention may be made of pyrazolo[1,5-a]pyridin-3-ylamine; 2-acetylaminopyrazolo[1,5-a]pyridin-3-ylamine; 2-morpholin-4-ylpyrazolo[1,5-a]pyridin-3-ylamine; 3-aminopyrazolo[1,5-a]pyridine-2-carboxylic acid; 2-methoxypyrazolo[1,5-a]pyridin-3-ylamino; (3-aminopyrazolo[1,5-a]pyridin-7-yl)methanol; 2-(3-aminopyrazolo[1,5-a]pyridin-5-yl)ethanol; 2-(3-aminopyrazolo[1,5-a]-pyridin-7-yl)ethanol; (3-aminopyrazolo[1,5-a]pyridin-2-yl)methanol; 3,6-diaminopyrazolo[1,5-a]pyridine; 3,4-diaminopyrazolo[1,5-a]pyridine; pyrazolo[1,5-a]-pyridine-3,7-diamine; 7-morpholin-4-ylpyrazolo[1,5-a]pyridin-3-ylamine; pyrazolo[1,5-a]pyridine-3,5-diamine; 5-morpholin-4-ylpyrazolo[1,5-a]pyridin-3-ylamine; 2-[(3-aminopyrazolo[1,5-a]pyridin-5-yl)(2-hydroxyethyl)amino]ethanol; 2-[(3-aminopyrazolo[1,5-a]pyridin-7-yl) (2-hydroxyethyl)amino]ethanol; 3-aminopyrazolo-[1,5-a]pyridin-5-ol; 3-aminopyrazolo[1,5-a]pyridin-4-ol; 3-aminopyrazolo[1,5-a]pyridin-6-ol; 3-aminopyrazolo[1,5-a]pyridin-7-ol; and acid or base addition salts thereof.

Among the pyrimidine derivatives, mention may be made of the compounds described, for example, in patents DE 2359399; JP 88-169571; JP 05-63124 and EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and pyrazolopyrimidine derivatives such as those mentioned in patent application FR-A-2750048, and among which mention may be made , for example, of pyrazolo[1,5-a]pyrimidine-3,7-diamine; 2,5-dimethylpyrazolo[1,5-a]-pyrimidine-3,7- diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[( 7-aminopyrazolo[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5-N7,N7-tetramethylpyrazolo[1,5-a]pyrimidine-3,7-diamine, 3-amino-5-methyl-7-imidazolylpropylaminopyrazolo[1,5-a]-pyrimidine, and acid addition salts thereof and tautomeric forms thereof, when a tautomeric equilibrium exists.

Among the pyrazole derivatives, mention may be made of the compounds described, for example, in patents DE 3843892 and DE 4133957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and acid addition salts thereof.

In the composition disclosed herein, the at least one oxidation base is generally present in an amount ranging, for example, from 0.001% to 10% by weight, such as from 0.005% to 6% by weight, of the total weight of the dye composition.

As the couplers, mention may, for example, be made of meta-phenylenediamines, meta-aminophenols, meta-diphenols, naphthalene couplers, heterocyclic couplers, and addition salts thereof.

By way of example, mention may be made of 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-7dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxy-benzomorpholine, 3,5-diamino-2,6-dimethoxypyridine, 1-N-(β-hydroxyethyl)amino-3,4-methylenedioxybenzene, 2,6-bis(β-hydroxyethylamino)toluene, and acid addition salts thereof.

In the composition disclosed herein, the at least one coupler is generally present in an amount ranging, for example, from 0.001% to 10% by weight, such as from 0.005% to 6% by weight, of the total weight of the dye composition.

In general, the addition salts of the oxidation bases and the couplers that can be used herein are, for example, chosen from addition salts with an acid, such as hydrochlorides, hydrobromides, sulphates, citrates, succinates, tartrates, lactates, tosylates, benzenesulphonates, phosphates and acetates, and addition salts with a base, such as sodium hydroxide, potassium hydroxide, aqueous ammonia, amines or alkanolamines.

The dye composition disclosed herein may also comprise at least one direct dye that may, for example, be chosen from nitro dyes of the benzene series, azo direct dyes and methine direct dyes. These direct dyes may be non-ionic, anionic or cationic in nature.

The composition may also comprise at least one auxiliary film-forming agent such as a plasticizer and/or an agent for facilitating the formation of a film of the at least one elastomeric film-forming polymer on the keratin materials, the function of which is to modify the properties of the at least one elastomeric film-forming polymer. Such an auxiliary film-forming agent may be chosen from all the compounds known to those skilled in the art as being capable of performing the desired function, and may, for example, be chosen from plasticizers and coalescence agents. The at least one elastomeric film-forming polymer, optionally combined with at least one auxiliary film-forming agent chosen from plasticizers and agents for facilitating film formation, is capable of forming a film, after evaporation of the cosmetic medium. This evaporation can be carried out in the open air or by providing heat, for example, using a dryer.

As an example of the at least one auxiliary film-forming agent chosen from plasticizers and agents for facilitating film formation on keratin materials, use may be made of those described in document FR-A-2 782 917. For example, the at least one auxiliary film-forming agent is chosen from the common plasticizers and coalescence agents, such as:
glycols and their derivatives, such as diethylene glycol ethyl ether, diethylene glycol methyl ether, diethylene glycol butyl ether, diethylene glycol hexyl ether, ethylene glycol ethyl ether, ethylene glycol butyl ether, ethylene glycol hexyl ether, and pentylene glycol,
glyceryl esters,
propylene glycol derivatives, such as propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether,
acid esters, for example, carboxylic acid esters, such as citrates, phthalates, adipates, carbonates, tartrates, phosphates and sebacates, and
mixtures thereof.

The amount of the at least one auxiliary film-forming agents can be chosen by those skilled in the art based on their general knowledge, so as to obtain a polymeric system (elastomeric polymers+auxiliary film-forming agents) that results in a film having the desired mechanical properties, while at the same time allowing the composition to conserve the desired cosmetic properties. In practice, this amount ranges from 0.01% to 25% by weight, such as from 0.01% to 15% by weight, of the total weight of the composition.

The dye composition disclosed herein may also comprise at least one adjuvant chosen from various adjuvants conventionally used in compositions for dyeing hair, such as anionic, cationic, non-ionic, amphoteric or zwitterionic surfactants or mixtures thereof, anionic, cationic, non-ionic, amphoteric or zwitterionic polymers or blends thereof, mineral or organic thickeners, antioxidants, penetrating agents, sequestering agents, fragrances, buffers, dispersants, conditioning agents such as, for example, volatile or non-volatile silicones, film-forming agents other than those of the invention, and in particular non-ionic, cationic, anionic or amphoteric fixing polymers, preserving agents or opacifiers.

The medium suitable for the coloration, also called dye support, generally consists of water or a mixture of water and at least one organic solvent so as to solubilize the compounds that are not sufficiently water-soluble. As an organic solvent, mention may, for example, be made of C₁-C₄ lower alkanols, such as ethanol and isopropanol; polyols and polyol ethers, such as 2-butoxyethanol, propylene glycol, propylene glycol monomethyl ether, diethylene glycol monoethyl ether and monomethyl ether, and also aromatic alcohols such as benzyl alcohol or phenoxyethanol, and mixtures thereof.

The solvents are present in an amount ranging, for example, from 1% to 40% by weight, such as from 5% to 30% by weight, relative to the total weight of the dye composition.

The pH of the dye composition disclosed herein ranges, for example, from 3 to 12, such as from 5 to 11. It can be adjusted to the desired value by acidifying or basifying agents normally used in the dyeing of keratin fibers, or by conventional buffer systems.

Among the acidifying agents, mention may be made, by way of example, of inorganic or organic acids such as hydrochloric acid, orthophosphoric acid, sulphuric acid, or carboxylic acids such as acetic acid, tartaric acid, citric acid or lactic acid, or sulphonic acids.

Among the basifying agents, mention may, by way of example, be made of aqueous ammonia, alkali metal carbonates, alkanolamines such as mono-, di- and triethanolamines and derivatives thereof, sodium hydroxide or potassium hydroxide, and the compounds of formula (II) below:

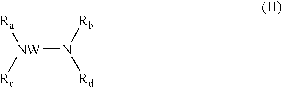

wherein W is a propylene residue optionally substituted with a hydroxyl group or a C₁-C₄ alkyl radical; and Rₐ, R_{b}, R_{c} and R_{d}, which may be identical or different, are each a hydrogen atom, or a C₁-C₄ alkyl or C₁-C₄ hydroxyalkyl radical.

The dye composition disclosed herein may be in various forms, such as in the form of liquids, creams or gels, or in any other form that is suitable for dyeing keratin fibers, such as human hair.

The method disclosed herein comprises applying the composition as defined above to the fibers in the presence of at least one oxidizing agent for a sufficient amount of time to develop the desired coloration. The color may be revealed at acid, neutral or alkaline pH and the at least one oxidizing agent may be added to the composition disclosed herein at the very moment of use or it may be used from an oxidizing composition containing it, applied simultaneously or sequentially to the composition disclosed herein.

In one embodiment, the composition disclosed herein is mixed, for example, at the time of use, with a composition comprising, in a medium suitable for dyeing, at least one oxidizing agent, wherein the at least one oxidizing agent is present in an amount sufficient to develop a coloration. The mixture obtained is then applied to the keratin fibers. After an application time ranging from 3 to 50 minutes, such as from 5 to 30 minutes, the keratin fibers are rinsed, washed with shampoo, rinsed again, and then dried.

The conventional oxidizing agents used are, for example, hydrogen peroxide, urea peroxide, alkali metal bromates, persalts such as perborates and persulphates, peracids and oxidase enzymes, among which mention may be made, for example, of peroxidases, 2-electron oxidoreductases such as uricases, and 4-electron oxygenases such as laccases. In one embodiment, hydrogen peroxide is used.

The oxidizing composition may also comprise at least one adjuvant chosen from various adjuvants conventionally used in compositions for dyeing the hair and as defined above.

The pH of the oxidizing composition comprising the at least one oxidizing agent is such that, after mixing with the dye composition, the pH of the resulting composition applied to the keratin fibers ranges, for example, from 3 to 12, such as from 5 to 11. It may be adjusted to the desired value by acidifying or basifying agents normally used in the dyeing of keratin fibers and as defined above.

The ready-to-use composition that is finally applied to the keratin fibers may be in various forms, such as in the form of liquids, creams or gels, or in any other form suitable for dyeing keratin fibers, for example, human hair.

The dye composition disclosed herein may be in various forms, such as in the form of liquids, creams or gels, or in any other form suitable for dyeing keratin materials, for example, human hair.

Those skilled in the art will take care to choose this or these optional additional compound(s) in such a way that the advantageous properties intrinsically associated with the oxidation dyeing composition disclosed herein are not, or are not substantially, impaired by the addition(s) envisaged.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The following examples illustrate the present disclosure without, however, limiting the scope thereof.

### EXAMPLES

The amounts are given as percentages by mass, and A.M. means active material.

### Example 1

A dye composition was prepared, comprising:

| Polyurethane (NMDEA¹⁾/PTMO 2900²⁾/IPDI³⁾ - 3/1/4) | 2% | AM |
|---|---|---|
| Para-phenylenediamine | 0.25 | g |
| Resorcin | 0.25 | g |
| Dyeing medium | (*) | |
| Demineralized water | qs 100 | g |

¹⁾N-methyldiethanolamine
²⁾poly(tetramethylene oxide) having a weight-average mass of 2900
³⁾isophorone diisocyanate

The polyurethane was prepared according to the method of synthesis described in French patent application FR 2 815 350.

The film obtained with the polyurethane had the following characteristics, measured as above:
ε_{b}=1500%
Rᵢ=82%
R₃₀₀=92%
water-soluble at at least 10 g per liter.

Dyeing Medium (*)

| Oleyl alcohol polyglycerolated with | 4 | g |
|---|---|---|
| 2 mol of glycerol | | |
| Oleyl alcohol polyglycerolated with | 5.69 | g A.M. |
| 4 mol of glycerol (78% A.M.) | | |
| Oleic acid | 3.0 | g |
| Oleylamine comprising 2 mol of | 7 | g |
| ethylene oxide, sold under the name | | |
| Ethomeen O12 by the company Akzo | | |
| Diethylaminopropyl laurylamino- | 3 | g A.M. |
| succinamate, sodium salt, comprising | | |
| 55% A.M. | | |
| Oleyl alcohol | 5 | g |
| Oleic acid diethanolamide | 12 | g |
| Propylene glycol | 3.5 | g |
| Ethyl alcohol | 7.0 | g |
| Dipropylene glycol | 0.5 | g |
| Propylene glycol monomethyl ether | 9 | g |
| Sodium metabisulphite as an aqueous | 0.455 | g A.M. |
| solution comprising 35% A.M. | | |
| Ammonium acetate | 0.8 | g |
| Antioxidant, sequestering agent | qs | |
| Fragrance, preserving agent | qs | |
| Aqueous ammonia comprising 20% NH₃ | 10.2 | g |

At the time of use, the composition was mixed with an equal weight of 20-volumes aqueous hydrogen peroxide (6% by weight). The mixture obtained was applied to locks of natural grey hair comprising 90% white hairs. After application for 30 minutes, the locks were rinsed, washed with a standard shampoo, rinsed again, and then dried.

A dark blond coloration of the keratin fibers was thus obtained.

### Example 2

A dye composition was prepared, comprising:

| Polyurethane (NMDEA¹⁾/PTMO 2900²⁾/IPDI³⁾ - 3/1/4) | 2% | AM |
|---|---|---|
| Para-aminophenol | 0.4 | g |
| 2-Methyl-5-aminophenol | 0.5 | g |
| Dyeing medium | (*) | |
| Demineralized water | qs 100 | g |

¹⁾N-methyldiethanolamine
²⁾poly(tetramethylene oxide) having a weight-average mass of 2900
³⁾isophorone diisocyanate
Dye medium (*) was the same as in Example 1.

The composition was mixed with aqueous hydrogen peroxide and applied to the keratin fibers according to the method described in Example 1.

A dark coppery blond coloration of the fibers was thusobtained.

### Example 3

A dye composition was prepared, comprising:

| Polyurethane (NMDEA¹⁾/PTMO 2900²⁾/IPDI³⁾ - 3/1/4) | 2% | AM |
|---|---|---|
| 4,5-Diamino-1-hydroxyethylpyrazole, sulphate | 1.0 | g |
| 2-Methyl-5-aminophenol | 0.7 | g |
| Dyeing medium | (*) | |
| Demineralized water | qs 100 | g |

¹⁾N-methyldiethanolamine
²⁾poly(tetramethylene oxide) having a weight-average mass of 2900
³⁾isophorone diisocyanate
Dye medium (*) was the same as in Example 1.

The composition was mixed with aqueous hydrogen peroxide and applied to the keratin fibers according to the method described in Example 1.

A light coppery chestnut brown coloration of the fibers was thus obtained.

What is claimed is:

1. A dyeing composition comprising, in a medium suitable for dyeing, at least one dye precursor and at least one elastomeric film-forming polymer wherein the film obtained by drying the at least one elastomeric film-forming polymer, at ambient temperature and at a relative humidity of 55%±5%, has properties comprising:
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%.

2. The composition according to claim 1, wherein the at least one elastomeric film-forming polymer is soluble in an aqueous or aqueous-alcoholic medium.

3. The composition according to claim 1, wherein the at least one elastomeric film-forming polymer is such that, at a relative humidity ranging from 30% to 80%, the elongation at break of the film obtained ranges from 400% to 1200% and/or the instantaneous recovery ranges from 57% to 93%.

4. The composition according to claim 1, wherein the at least one elastomeric film-forming polymer is present in an amount ranging from 0.05% to 20% by weight of the total weight of the composition.

5. The composition according to claim 4, wherein the at least one elastomeric film-forming polymer is present in an amount ranging from 0.1% to 15% by weight of the total weight of the composition.

6. The composition according to claim 1, wherein the at least one elastomeric film-forming polymer is chosen from polyurethanes, polyvinyl alcohols, and polymers comprising at least one (meth)acrylic unit.

7. The composition according to claim 1, wherein the at least one dye precursor is chosen from oxidation bases.

8. The composition according to claim 7, wherein the oxidation bases are chosen from para-phenylenediamines, bisphenylalkylenediamines, para-aminophenols, ortho-aminophenols and heterocyclic bases.

9. The composition according to claim 7, wherein the oxidation bases are chosen from para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene, 3-hydroxy-1-(4′-aminophenyl)pyrrolidine, and acid addition salts thereof.

10. The composition according to claim 8, wherein the bisphenylalkylenediamines are chosen from N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′amino-3′-methylphenyl)ethylenediamine, 1,8-bis(2,5-diaminophenoxy)-3,6-dioxaoctane, and acid addition salts thereof.

11. The composition according to claim 8, wherein the para-aminophenols are chosen from para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and acid addition salts thereof.

12. The composition according to claim 8, wherein the ortho-aminophenols are chosen from 2-aminophenol, 2-amino-5-methylphenol, 2-amino-6-methylphenol, 5-acetamido-2-aminophenol, and acid addition salts thereof.

13. The composition according to claim 8, wherein the heterocyclic bases are chosen from pyridine derivatives, pyrimidine derivatives and pyrazole derivatives.

14. The composition according to claim 13, wherein the pyridine derivatives are chosen from 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, pyrazolo[1,5-a]pyridin-3-ylamine; 2-acetylaminopyrazolo[1,5-a]pyridin-3-ylamine; 2-morpholin-4-ylpyrazolo[1,5-a]pyridin-3-ylamine; 3-aminopyrazolo[1,5-a]pyridine-2-carboxylic acid; 2-methoxypyrazolo[1,5-a]pyridin-3-ylamino; (3-aminopyrazolo[1,5-a]-pyridin-7-yl)methanol; 2-(3-aminopyrazolo[1 ,5-a]pyridin-5-yl)ethanol; 2-(3-amino-pyrazolo[1,5-a]pyridin-7-yl)ethanol; (3-aminopyrazolo[1,5-a]pyridin-2-yl)methanol; 3,6-diaminopyrazolo[1,5-a]pyridine; 3,4-diaminopyrazolo[1,5-a]pyridine; pyrazolo[1,5-a]-pyridine-3,7-diamine; 7-morpholin-4-ylpyrazolo[1,5-a]pyridin-3-ylamine; pyrazolo[1,5-a]pyridine-3,5-diamine; 5-morpholin-4-ylpyrazolo[1,5-a]pyridin-3-ylamine; 2-[(3-aminopyrazolo[1,5-a]pyridin-5-yl) (2-hydroxyethyl)amino]ethanol; 2-[(3-aminopyrazolo[1,5-a]pyridin-7-yl)(2-hydroxyethyl)amino]ethanol; 3-aminopyrazolo-[1,5-a]pyridin-5-ol; 3-aminopyrazolo[1,5-a]pyridin-4-ol; 3-aminopyrazolo[1,5-a]pyridin-6-ol; 3-aminopyrazolo[1,5-a]pyridin-7-ol; and acid or base addition salts thereof.

15. The composition according to claim 13, wherein the pyrimidine derivatives are chosen from 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethyl-pyrazolo[1,5-a]pyrimidine-3,7-diamine, 2,6-dimethylpyrazolo[1,5-a]pyrimidine-3,7-diamine, 2,5-N7, N7-tetramethylpyrazolo[1,5-a]pyrimidine-3,7-diamine, 3-amino-5-methyl-7-imidazolylpropylaminopyrazolo[1,5-a]pyrimidine, and acid addition salts thereof and tautomeric forms thereof, when a tautomeric equilibrium exists.

16. The composition according to claim 13, wherein the pyrazole derivatives are chosen from 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethyl-pyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-tri-aminopyrazole, 3,5-diamino-1-methyl-4-methylaminopyrazole, 3,5-diamino-4-(β-hydroxyethyl)amino-1-methylpyrazole, and acid addition salts thereof.

17. The composition according to claim 7, wherein the at least one dye precursor chosen from oxidation bases is present in an amount ranging from 0.001% to 10% by weight of the total weight of the composition.

18. The composition according to claim 1, wherein the at least one dye precursor is chosen from couplers chosen from meta-phenylenediamines, meta-aminophenols, meta-diphenols, naphthols, indole, indoline, pyridine and indazole couplers, pyrazolo[1,5-b]-1,2,4-triazole, pyrazolo[3,2-c]-1,2,4-triazole, benzimidazole, benzothiazole, benzoxazole, 1,3-benzodioxole and pyrazolones, and acid addition salts thereof.

19. The composition according to claim 18, wherein the at least one dye precursor is chosen from couplers chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, α-naphthol, 2-methyl-1-naphthol, 6-hydroxyindole, 4-hydroxyindole, 4-hydroxy-N-methylindole, 6-hydroxyindoline, 6-hydroxybenzomorpholine, 3,5-diamino-2,6-dimethoxypyridine, 1-N-(β-hydroxyethyl)amino-3,4-methylenedioxybenzene, 2,6-bis(13-hydroxyethylamino)toluene, 2,6-dihydroxy-4-methylpyridine, 1-H-3-methylpyrazol-5-one, 1-phenyl-3-methylpyrazol-5-one, and acid addition salts thereof.

20. The composition according to claim 18, wherein the at least one dye precursor chosen from couplers is present in an amount ranging from 0.0001 to 10% by weight of the total weight of the composition.

21. The composition according to claim 1, further comprising at least one oxidizing agent.

22. The composition according to claim 21, wherein the at least one oxidizing agent is chosen from hydrogen peroxide, urea peroxide, alkali metal bromates, persalts, peracids and oxidase enzymes.

23. A method for dyeing a keratin material, comprising applying to the keratin material a composition, comprising, in a medium suitable for dyeing, at least one dye precursor and at least one elastomeric film-forming polymer wherein the film obtained by drying the at least one elastomeric film-forming polymer, at ambient temperature and at a relative humidity of 55%±5%, has properties comprising:
(a) an elongation at break (ε_{b}) of greater than or equal to 800%,
(b) an instantaneous recovery (Rᵢ) at least equal to 75%, after an elongation of 150%, and
(c) a recovery (R₃₀₀) at 300 seconds of greater than 80%, in the presence of at least one oxidizing agent for a period of time sufficient to color the keratin material.

24. The method according to claim 23, wherein the keratin material is keratin fibers.
